(19) United States
(12) Patent Application Publication
(10) Pub. No.: US 2017/0220740 A1
(43) Pub. Date: Aug. 3, 2017

(54) SYSTEM FOR DELIVERING PERSONALIZED HEALTH CARE

(71) Applicant: Paul S. D'URSO, Richmond, Victoria (AU)

(72) Inventor: Paul S. D'URSO, Richmond, Victoria (AU)

(21) Appl. No.: 15/501,380

(22) PCT Filed: Aug. 5, 2015
(86) PCT No.: PCT/AU2015/050443

§ 371 Date: Feb. 2, 2017

(30) Foreign Application Priority Data
Aug. 5, 2014 (AU) 2014100879
Aug. 5, 2014 (AU) 2014100880
Jul. 25, 2015 (AU) 2015902963

(51) Int. Cl. G06F 19/00; A61B 6/00; A61B 5/00; A61B 6/03

(52) U.S. Cl. CPC G06F 19/321; G06F 19/3481; G06F 19/328; A61B 6/03; A61B 6/563; A61B 5/4836

(57) ABSTRACT

There are provided systems for delivering personalized health care. The systems utilize a server computer system for design, storage and delivery of patient specific medical solutions, particularly surgical procedures. The systems allow information to be disseminated to key stakeholders quickly and effectively.

### FIELD

The present disclosure relates generally to a system for delivering personalized health care. More specifically, the disclosure is directed to a server computer system for delivering personalized health care which facilitates design of, access to, and authorization of patient specific medical solutions based on patient specific medical information.

### BACKGROUND

The medical device industry is experiencing an evolutionary transformation in which diagnostic and therapeutic interventions are increasingly personalized, with patient-specific solutions aligned to individual patient conditions, improving therapeutic benefits and in most cases, lowering overall costs. To support this transformation, medical device companies must evolve their business models to support an engineered-to-order approach. Three dimensional printing can assist medical device companies with rapid prototyping, as well as the full production of specific types of products. However, there has been less focus on the concept of customization—the provision of patient specific solutions, for example the custom-tailoring of a medical device to perfect fit with the unique elements of a patient's body. Such customization generates regulatory, communication and coordination difficulties between key stakeholders, for example physicians, point of care facilities, insurance companies, and government departments.

Therefore, a need exists for systems and methods which facilitate the development and rapid dissemination of patient specific medical solutions to key stakeholders and which address the above difficulties.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

### SUMMARY

In one aspect there is provided a server computer system comprising:

one or more processors;

one or more memories coupled to the processor;

one or more network interfaces coupled to the processor;

wherein the memory contains executable instructions configured to cause the system to implement a method comprising:

(a) receiving via the network interface patient specific information comprising medical imaging data;

(b) processing said information to design at least one patient specific medical solution; and

(c) providing access to the at least one patient specific medical solution via the network interface, to one or more members of a health care network.

The system may be used to deliver personalized health care.

The server computer system may be cloud based.

The server computer system may be a distributed system.

The method may also comprise providing access to the patient specific information via the network interface, to one or more members of a health care network.

The medical solution may comprise the design of one or more patient specific medical devices. The medical solution may comprise the selection of one or more compatible off-the-shelf medical devices. The medical solution may comprise a combination of the design of patient specific medical devices and the selection of compatible off-the-shelf medical devices.

The medical solution may comprise a surgical solution. The surgical solution may comprise at least one surgical procedure.

The at least one surgical procedure may comprise an orthopedic procedure. Exemplary, but not limiting, orthopedic procedures include spine surgery and joint replacement.

The at least one surgical procedure may comprise a minimally invasive surgical procedure.

The members of the healthcare network may be one or more of physicians, point of care facilities, insurance companies, medical device manufactures and government departments.

Processing the information may comprise computer aided design.

The medical imaging data may comprise three-dimensional medical imaging data.

The medical imaging data may comprise computed tomography data.

The at least one network interface may provide access to, for example, medical images, three dimensional printed biomodels and medical device design.

The at least one network interface may allow a selection of medical devices and surgical instruments.

The server computer system may store patient specific information, medical solutions, cost data and logistic data in which are accessible via the network interface. The storage may in one or more searchable databases.

The server computer system may store data on the cost and availability of medical devices, which is accessible via the network interface.

The at least one network interface may further provide access to cost and/or logistic information.

The patient specific solution may comprise a combination of medical devices and instrumentation.

The server computer system may be licensed by point of care facilities, for example hospital networks, to manage patient specific solutions, including devices and prosthetics. Advantageously, this creates efficiencies and cost savings.

A physician may use medical imaging data to pre-plan and design, and select the at least one patient specific solution. The solution may be quickly transmitted using the method implemented on the server computer system to members of the healthcare network so that the solution may be efficiently authorised, manufactured and delivered in a time critical manner.

The method may allow a physician to integrate of all of the information and create a solution that is then communicated to stakeholders and delivered.

Access to up to date insurance status and medical device availability may be accessed via the network interface.

A physician may book surgical procedures at a point of care facility via the network interface.

A physician may access, for example, three dimensional images, three dimensional and printed biomodels via the network interface.

A physician may design implants and instruments, and select devices and instruments via the network interface.

In one embodiment, a physician, via the network interface, may only access the device companies that the point of care facility approves. That is, access may only be granted to preferred providers.

A point of care facility may regulate a physician's options via the network interface.

Insurance companies and/or a relevant government department may monitor physician activity and deny or approve the patient specific solutions from the outset, via the network interface, therefore improving efficiencies.

A device company may update inventory in real time via the network interface.

A device company or insurer may advertise to physicians and point of care facilities notifying them of new developments and/or of relevant information via the network interface.

A point of care facility may regulate access of certain information to other members of the healthcare network via the network interface.

A physician may apply to a point of care facility to have companies placed on the system via the network interface.

Device companies may subscribe to the system to receive orders from physicians as ‘preferred’ orders with advanced warning, via the network interface.

Physicians and point of care facilities may utilize the system to innovate and develop ideas. Commercial innovations may be added via the network interface. These may generate royalties for physicians and point of care facilities.

Insurance companies may utilize the system to regulate liabilities. The insurer may receive advanced warning of liabilities at the time of medical procedure booking rather than well after the medical procedure has been performed. The insurer may authorize liability for patient specific solutions and ex gratia requests in a timely fashion upon notification. The insurers may pay a subscription to use the system.

The computer server system may also include an audit function. Through the network interface, a point of care facility may capture information in the system regarding a medical procedure and enter the information into a database which may reside on the server to track the patient's “episode of care”. This may include, for example, what medical procedure was actually performed, how long it took, what was actually used from the patient specific solution, time of hospital stay, complications, and so forth. This information may then be relayed via the system back to the physicians, insurance company or government department. Such audit information may then be used to assess the efficiency of the system and identify opportunities for improvement or identify physicians that are not efficiently planning procedures.

In this way, the audit function provides a feedback mechanism to insurance companies and point of care facilities. The effectiveness of the system may then be monitored and improved.

The system may comprise any one or more of the hereinbefore disclosed embodiments in any combination.

There is also provided a method for delivering personalized health care comprising the following steps:

(a) obtaining patient specific information using medical imaging;

(b) relaying said information to a server computer system;

(c) designing via the server computer system at least one patient specific medical solution; and

(d) accessing via a network interface, said solution.

The solution may comprise the design of one or more patient specific medical devices. The solution may comprise the selection of compatible off-the-shelf medical devices. The solution may comprise a combination of the design of patient specific medical devices and the selection of compatible off-the-shelf medical devices.

The solution may comprise a surgical solution. The surgical solution may comprise at least one surgical procedure.

The at least one surgical procedure may comprise an orthopedic procedure. Exemplary, but not limiting, orthopedic procedures include spine surgery and joint replacement.

The at least one surgical procedure may comprise a minimally invasive surgical procedure.

Access to said information and said solution may be by one or more of physicians, point of care facilities, insurance companies, medical device manufactures and government departments.

The method may further comprise the step of ordering via the network interface the at least one patient specific medical solution based on the design.

The method may further comprise the step of manufacturing the at least one patient specific medical solution.

The method may further comprise the step of providing the at least one patient specific medical solution to a point of care facility.

The method may further comprise the step of executing the at least one patient specific medical solution at a point of care facility.

The design of one or more patient specific medical devices may comprise computer aided design.

The medical imaging may comprise three-dimensional medical imaging.

The patient specific information may comprise at least one three- dimensional medical image.

The medical imaging may comprise computed tomography.

The patient specific medical solution may comprise a kit of parts. The kit may comprise elements necessary to execute the medical solution.

Accordingly, there is also provided a kit of parts for delivering a patient specific medical solution, said kit comprising:

(a) one or more medical devices; and

(b) one or more medical instruments.

The kit of parts may further comprise packaging adapted to contain and maintain one or medical devices and/or one or more medical instruments in a sterilized condition prior to delivery of the solution.

There is also provided a kit of parts for delivering a patient specific medical solution according to any one of the hereinbefore disclosed systems or methods, said kit comprising:

(a) one or more medical devices; and

(b) one or more medical instruments.

The kit of parts may further comprise packaging adapted to contain and maintain one or medical devices and/or one or more medical instruments in a sterilized condition prior to delivery of the solution.

The kit of parts may comprise more than one kit of parts or two or more kits of parts. For example, medical devices, originating from a medical device company, may be in one kit of parts, and standard medical instrumentation may be in another.

The one or more medical devices may comprise one or more patient specific medical devices.

The patient specific medical solution may comprise at least one surgical solution.

In any one of the hereinbefore disclosed embodiments the medical devices may be manufactured using rapid prototype technology.

In any of the hereinbefore disclosed embodiments the medical devices may be manufactured using three-dimensional printing.

In any of the herein disclosed embodiments a physician may be a surgeon.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

### BRIEF DESCRIPTION OF THE DRAWINGS

FIG. 1 illustrates a server computer system according an embodiment of the present disclosure.

FIG. 2 is a flow chart of a method utilising a system according to an embodiment of the present disclosure.

FIG. 3 is a flow chart of a method utilising a system according to another embodiment of the present disclosure.

FIG. 4 is a flow chart of a method utilising a system according to another embodiment of the present disclosure.

### DESCRIPTION OF EXEMPLARY EMBODIMENTS

Before the present systems, devices and methods are disclosed and described, it is to be understood that unless otherwise indicated this disclosure is not limited to specific systems, devices, components, designs, methods, or the like, as such may vary, unless otherwise specified. It is also to be understood that the terminology used herein is for the purpose of describing particular embodiments only and is not intended to be limiting.

It must also be noted that, as used in the specification and the appended claims, the singular forms ‘a’, ‘an’ and ‘the’ include plural referents unless otherwise specified. Thus, for example, reference to ‘a device’ may include more than one devices, and the like.

Disclosed herein are server computer systems and related methods for the delivery of patient specific information, solutions, devices and prosthetics into healthcare networks.

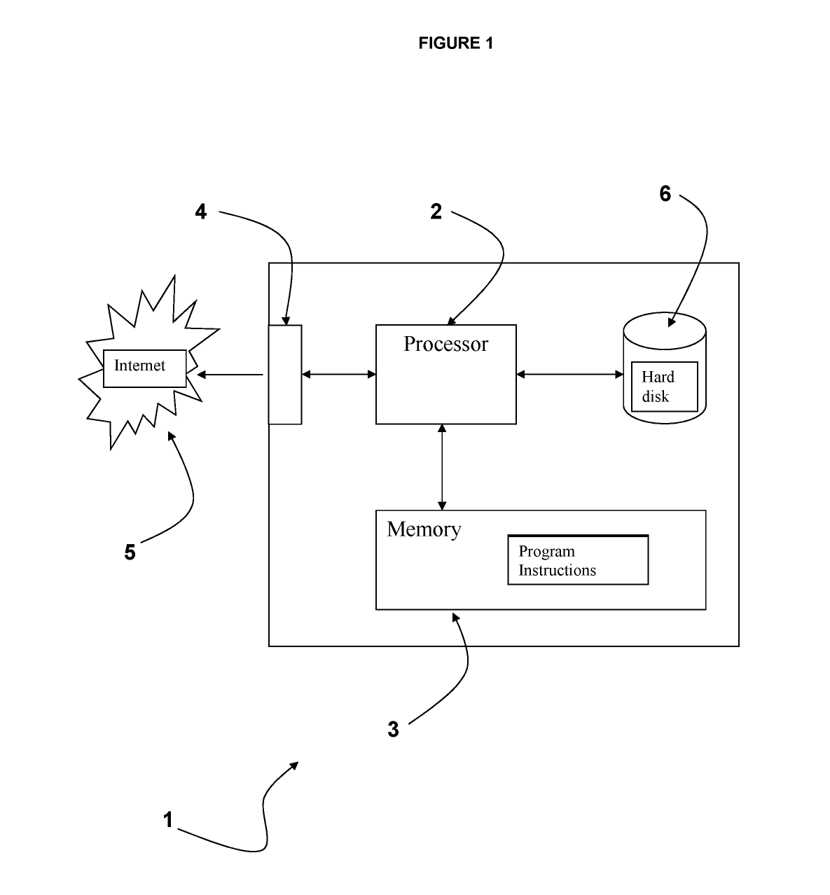

Referring to FIG. 1, a server computer system according to an embodiment of the present disclosure is illustrated. The system comprises processor (2) coupled to memory (3). The memory contains executable instructions, for example, to implement computer aided design or interrogate databases. The processor is coupled to a network interface (4) accessible through the internet (5). Hard disk (6) comprises programs, storage and data.

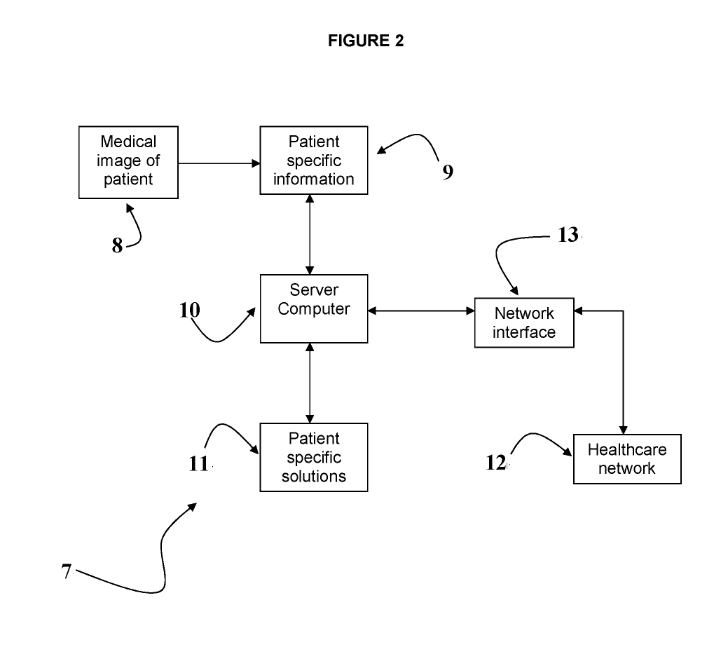

Referring to FIG. 2, method (7) is illustrated. Medical image (8) of a patient provides patient specific information (9). The information (9) is relayed to server computer (10) which processes the information to provide patient specific solutions (11). Information (9) and solutions (11) are accessible by healthcare network (12) via network interface (13).

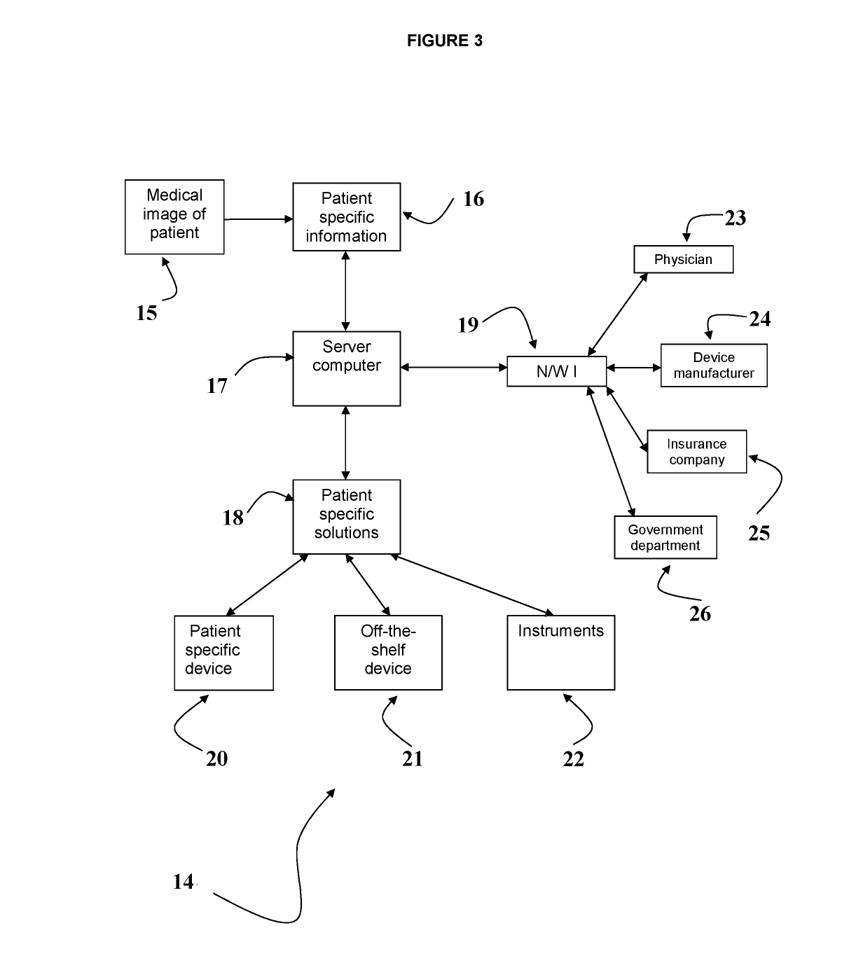

Referring to FIG. 3, method (14) is illustrated. Medical image (15) of a patient provides patient specific information (16). The information (16) is relayed to server computer (17) which processes the information to provide patient specific solutions (18). The patient specific solutions may be in the form of the design of patient specific devices (20), a selection of off-the-shelf devices (21) and instruments (22). Information (16) and solutions (18) are accessible via network interface (19) by key stakeholders, illustrated as physicians (23), device manufacturers (24), insurance companies (25) and government departments (26).

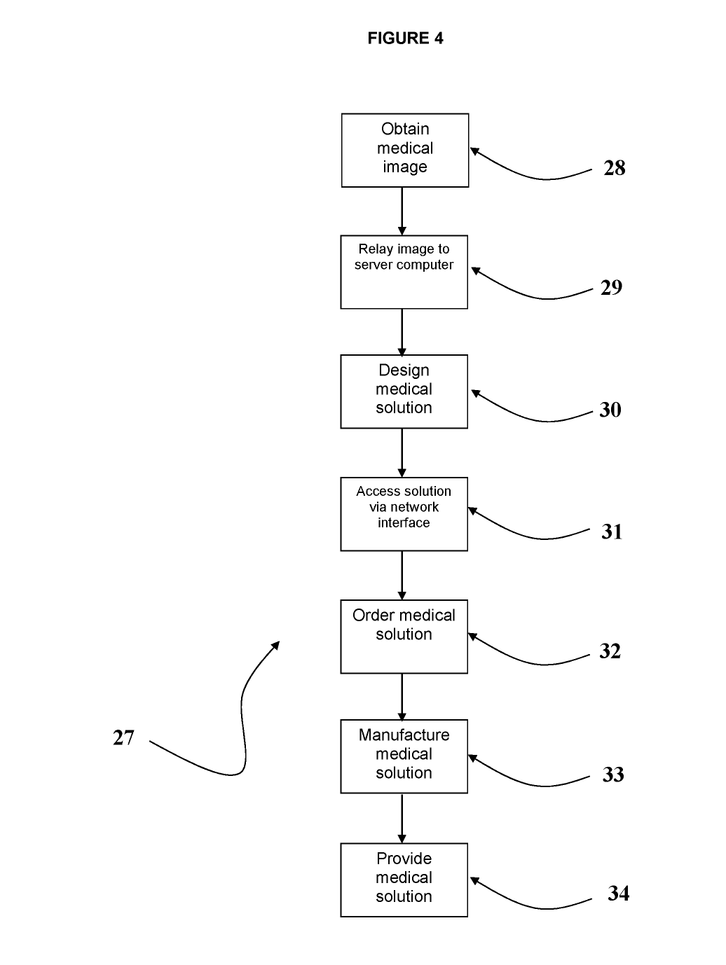

Referring to FIG. 4, method (27) is illustrated. A medical image of a patient is obtained (28) and relayed (29) to a server computer. The server computer hosts computer aided design programs which allow the design (30) at least one patient specific solution which is subsequently accessible (31) via the network interface. The solution may then be ordered (32), manufactured (33) and the solution then provided (34) to, for example, a point of care facility.

A feature of the server computer system of the present disclosure is the interactive communication between the various stakeholders. For example, device manufacturer (24) may access device designs (20) via network interface (19), and provide cost information on device manufacture to the system. Cost authorization by, for example, an insurance company (25) may be uploaded via the network interface and the device subsequently manufactured by the device company. Communications and outcomes may be viewed via the network interface by the physician.

In an exemplary embodiment the system may be applied to the provision of spinal surgery.

In regard to the lumbar spine, minimally invasive surgical techniques may be applied to the operation of transforaminal lumbar interbody fusion (TLIF). A minimally invasive transforaminal lumbar interbody fusion can be performed by four small 2 cm incisions in the lumbar region. Into the incisions it us used to place pedicle screws, one of the incisions is used to place a retractor system so that the surgeon can see the anatomical elements such as the nerve roots and intervertebral disc. The surgeon uses a retractor to perform laminectomy to decompress the associated exiting nerve roots, perform lumbar discectomy and prepare the interbody space and then place bone graft and an interbody cage to facilitate interbody fusion. Minimally invasive TLIF has evolved to a stage where a surgery can be performed in a highly repeatable fashion with a high degree of safety and reproducibility of outcome. Minimally invasive TLIF is a suitable procedure to treat any level in the lumbar spine for most of the common pathologies that the spinal surgeon encounters.

In order to evolve this procedure to a greater level of ergonomic efficiency and safety and best possible patient outcomes, three dimensional printing and rapid prototype technology may be utilized to facilitate these outcomes.

The systems and methods of the present disclosure facilitate minimally invasive spinal surgery in a way that provides substantial improvement in efficiency and cost effectiveness. The present systems and methods require the surgeon to be interactive in such a way that surgical planning can be predetermined and a patient specific solution can be pre-packaged to facilitate lumbar interbody fusion. The system and method of the present disclosure have been developed so that surgeons with only basic equipment can perform state of the art lumbar interbody fusion surgery at any standard hospital facility throughout the world.

The system of the present disclosure packages the spinal device technology into a kit of parts comprising a sterilized set of instruments and medical devices that can be easily transported and that are personalized to a particular patient and procedure.

In order to initiate TLIF using the system of the present disclosure the following are required:

1. Computed tomography scan of a patient's spine

2. A computer with internet access

3. A point of care facility with an operating theatre having an image intensifier and a radiolucent operating table.

With these basic requirements, a surgeon anywhere in the world may perform a minimally invasive transforaminal lumbar interbody fusion safely, efficiently and in a highly cost effective manor. The surgeon may have patient specific instruments and retractors and the highest quality prosthetic devices designed and delivered utilizing the system of the present disclosure to perform surgical intervention.

The patient specific devices and instruments may be assembled into one or more kits of parts .

In order to perform TLIF the kit of parts comprises the following:

1. A patient specific template to map skin incisions and assist with trajectory placement of Jam Shidi needles. An example of such a template is described in Applicant's co-pending application PCT/AU2015/050256, the disclosure of which is incorporated herein by reference in its entirety.

2. Nitolon K-wires

3. Jam Shidi needles

4. Tissue dilators with both electrocautery and neural monitoring capacity

5. Patient specific smart tube retractors for bone graft harvesting and interbody fusion cage delivery purposes. Such retractors will have incorporated within them fixation aids to the patient anatomy. Trajectory guides for K-wire assisted placement. Suction and irrigation channels, illumination mechanism, electrocautery and electrophysiological monitoring capacity. Patient specific contour matching is incorporated within the retractor. Examples of such retractors are described in Applicant's co-pending application PCT/AU2015/050258, the disclosure of which is incorporated herein by reference in its entirety.

6. Predetermined off-the-shelf devices including pedicle screws, interconnecting rods and lockers.

7. Intervertebral cage, preferably distractible in a 3-dimensional fashion. Examples of such cages are described in Applicant's co-pending application PCT/AU2015/050257, the disclosure of which is incorporated herein by reference in its entirety.

8. Bone substitute to repair the bone donor site and for interbody or posterolateral fusion augmentation.

9. Taps to assist with pedicle screw placement.

10. Disposable drill bit

11. Disposable bone mill attachment

12. Bone morphogenic protein in a predetermined volume

The kit of parts may be disposable and single use only. The components may be provided sterile. The components may be packaged in more than one kit of parts.

The system and methods of the present disclosure may be used to facilitate the surgery as follows:
Step 1
A surgeon recommends a TLIF and obtains a medical scan of the patient.
Step 2
The surgeon uses the system according to the present disclosure to plan surgery and order a combination of off-the-shelf devices and patient specific devices. A biomodel of the spine may also be provided to the surgeon prior to surgery to facilitate preplanning and characterisation of the components for the kit of parts.
Step 3
The system immediately communicates the order to the hospital, the insurer and the device companies.
Step 4
The kit of parts is assembled and transported to the hospital.
Step 5
The hospital prepares standard reusable instrument set, for example, comprising distraction instruments, osteotomes, power driver for placement of pedicle screws, bone mill for preparation of bone graft, and high speed electric drill.
Step 6
Hospital instruments and kit of parts are combined to perform the surgery.

While the foregoing description has focused on spinal surgery, it is contemplated that the systems and methods described herein may find use in a wide range of medical and particularly surgical applications.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the above examples are put forth so as to provide those skilled in the art with a complete disclosure and description of how to make and use the disclosed devices, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

1. A server computer system comprising:
one or more processors;
one or more memories coupled to the processor;
one or more network interfaces coupled to the processor;
wherein the memory contains executable instructions configured to cause the system to implement a method comprising:
(a) receiving via the network interface patient specific information comprising medical imaging data;
(b) processing said information to design at least one patient specific medical solution; and
(c) providing access to the at least one patient specific medical solution via the network interface, to one or more members of a health care network.

2. A system according to claim 1, wherein access is provided to the patient specific information via the network interface, to one or more members of a health care network.

3. A system according to claim 1 or claim 2, wherein the medical solution comprises the design of one or more patient specific medical devices.

4. A system according to claim 1, wherein the medical solution comprises the selection of compatible off-the-shelf medical devices.

5. (canceled)

6. A system according to claim 1, wherein the solution comprises a surgical solution.

7. A system according to claim 6, wherein the surgical solution comprises at least one surgical procedure.

8. A system according to claim 7, wherein the at least one surgical procedure comprises an orthopaedic procedure.

9. A system according to claim 8, wherein the at least one surgical procedure comprises a minimally invasive surgical procedure.

10. A system according to claim 1, wherein the members of the healthcare network are one or more of physicians, point of care facilities, insurance companies, medical device manufactures and government departments.

11. (canceled)

12. A system according to claim 1, wherein the at least one network interface provides access to medical images, three dimensional biomodels and medical device design.

13. A system according to claim 1, wherein the at least one network interface allows a selection of medical devices and surgical instruments to be made.

14. A system according to claim 1, wherein the at least one network interface further provides access to cost and/or logistic information.

15. (canceled)

16. (anceled)

17. (canceled)

18. A system according to claim 1, wherein the at least one network interface further provides access to patient insurance status and medical device availability.

19. A system according to claim 1, wherein the system further comprises an audit function.

20. A method for delivering personalized health care comprising the following steps:
(a) obtaining patient specific information using medical imaging;
(b) relaying said information to a server computer system;
(c) designing via the server computer system according to claim 1, at least one patient specific medical solution; and
(d) accessing via a network interface, said information and/or said solution.

21. A method according to claim 20, wherein the access to said information and/or said solution is by one or more of physicians, point of care facilities, insurance companies, medical device manufactures and government departments.

22. A method according to claim 20, further comprising the step of ordering at least one patient specific medical solution based on the design.

23. A method according to claim 20, further comprising the step of manufacturing the at least one patient specific medical solution.

24. A method according claim 20, further comprising the step of providing the at least one patient specific medical solution to a point of care facility.

25. A method according claim 20, wherein the medical solution comprises at least one surgical solution.

26. A kit of parts for delivering a patient specific medical solution, designated by the system according to claim 1, said kit comprising:
(a) one or more medical devices; and
(b) one or more medical instruments.

27. A kit of parts according to claim 26, further comprising packaging adapted to contain and maintain one or medical devices and/or one or more medical instruments in a sterilized condition prior to delivery of the solution.

28. (canceled)

29. A kit of parts according to claim 26, wherein the one or more medical devices comprises one or more patient specific medical devices.

30. (canceled)
